(19) United States
(12) Patent Application Publication
(10) Pub. No.: US 2015/0251105 A1
(43) Pub. Date: Sep. 10, 2015

(54) PROCESS OF SEPARATING UNSATURATED HYDROCARBONS FROM SATURATED HYDROCARBONS WTIH LOW ENERGY CONSUMPTION

(71) Applicant: GTC Technology US LLC, Houston, TX (US)

(72) Inventors: Venkata K. Ramanujam, Sugar Land, TX (US); Cole Nelson, Cypress, TX (US); Michael McCaulley, Sugar Land, TX (US)

(21) Appl. No.: 14/640,583

(22) Filed: Mar. 6, 2015

Related U.S. Application Data
(60) Provisional application No. 61/949,063, filed on Mar. 6, 2014.

(51) Int. Cl. B01D 3/14; C07C 7/00; C07C 7/04

(52) U.S. Cl. CPC B01D 3/146; C07C 7/04; C07C 7/005

(57) ABSTRACT

Apparatuses, systems and methods for separating highly pure unsaturated olefinic hydrocarbon stream with zero cooling water and or steam consumption, with minimum possible capital investment and uncompromised operational ease are disclosed herein from a mixture of hydrocarbon stream consisting of saturated and unsaturated hydrocarbons. Embodiments of the invention are directed to producing a hydrocarbon stream containing polymer, chemical grade ethylene, propylene, butylenes, isoprene, hexane stream which are of value in manufacturing chemicals, polymers, and rubbers. Embodiments of the process provided can be applied to concentrating ethylene, propylene, butylenes, cyclopentadiene, isoprene, 2 methyl butene, isopentane, and hexene.

### BACKGROUND OF THE INVENTION

A mixture of unsaturated hydrocarbons and saturated hydrocarbons are produced in the process of catalytic cracking, steam cracking, thermal cracking or dehydrogenation, hydrogenation process. Unsaturated hydrocarbons present in the mixture are building blocks for producing numerous chemicals, polymers, resins and rubbers. Hence it is highly desirable to have ultra pure unsaturated hydrocarbon stream. Variety of separation processes such as traditional fractional distillation, pressure swing adsorption or a combination of adsorption and distillation, extraction, extractive distillation, sponging distillation or combination of any of the above are used to separate the unsaturated and saturated hydrocarbons.

The relative volatility difference between the unsaturated hydrocarbon and its saturated counterpart is so low that the separation process invariably consumes excessive energy with excessive number of distillation trays. A number of alternative schemes such as high pressure distillation, low pressure distillation with heat pump, and divided wall column are suggested in the prior art to achieve the desired unsaturated hydrocarbon stream with minimum possible energy consumption.

### FIELD OF THE INVENTION

The claimed invention and the apparatuses and methods are intended to obtain unsaturated and saturated hydrocarbon streams in particular light olefins containing 2-6 carbons with minimum possible energy and investment and ease and reliable operation, one of which is otherwise sacrificed. Such apparatuses and methods would allow more efficient operation and system design and operating conditions.

Typically the unsaturated hydrocarbons and saturated hydrocarbons, particularly hydrocarbons containing 2 to 6 carbon atoms are separated using tall distillation columns, typically separated physically into two distillation columns. Alternately a heat pump system is used wherein, the overheads of the distillation column are compressed to high enough pressure to provide the required heat to the reboiler.

The present invention provides an improvement to the traditional distillation and heat pump system and other combinations by employing intermediate compressor and integrating the condenser and reboiler and exploiting the natural behavior of the unsaturated hydrocarbons and saturated hydrocarbons with respect to pressure to minimize the energy and capital and simultaneously keeping the operations simple and stable.

The present invention can be applied to systems consisting of unsaturated hydrocarbons and saturated hydrocarbons or isomers of saturated or unsaturated hydrocarbons or unsaturated hydrocarbons, boiling point difference between the components of which are less that 10° C., preferably less than 5° C. and separation of which require tall columns or extraction solvent or combination thereof.

### SUMMARY OF THE INVENTION

In various embodiments, system of apparatuses and operating conditions for separating the unsaturated hydrocarbons from saturated hydrocarbons, particularly hydrocarbons containing 2 to 6 carbon atoms are disclosed. The apparatuses comprise of a distillation column, a compressor, heat exchangers for reboiler and condenser systems, reflux drum and pumps for pumping the saturated hydrocarbon and unsaturated hydrocarbon.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 shows an illustrative unsaturated and saturated hydrocarbon separation system with low pressure stripper feed point; and

FIG. 2 shows an illustrative unsaturated and saturated hydrocarbon separation system with a high pressure rectification feed point.

### DETAILED DESCRIPTION OF EXEMPLARY EMBODIMENTS

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the ability of persons of ordinary skill in the relevant art.

Referring to the drawings in general, it will be understood that the illustrations are for the purpose of describing a particular embodiment of the disclosure and are not intended to be limiting thereto. Drawings are not necessarily to scale.

While most of the terms used herein will be recognizable to those of skill in the art, it should be understood, however, that when not explicitly defined, terms should be interpreted as adopting a meaning presently accepted by those of skill in the art.

“Unsaturated hydrocarbon,” as used herein, refers to, for example, light olefins such as ethylene, propylene, butylenes, methyl butenes and it's isomers, cis and, or trans pentene, hexanes and similar hydrocarbons. “Saturated Hydrocarbons” and as used herein, refers to Ethane, Propane, Butane, Pentane, Methyl Butane, Hexane and similar hydrocarbons.

In the catalytic cracking process in the thermal cracking, in the presence of steam of liquid fractions of petroleum, such as LPG, Naphtha, Diesel and heavies for production of ethylene and or propylene, or in the propane or Butane dehydrogenation process, a hydrocarbon liquid fraction consisting of unsaturated hydrocarbons and saturated hydrocarbons is produced. Also pyrolysis gasoline, a steam cracker hydrocarbon product consists of hydrocarbon components ranging from 4 carbon atoms to more than 10 carbon atoms. Among these, of importance to the field of innovation are mono olefins and diolefins such as ethylene, propylene, butylenes, cyclopentadiene (CPD), methyl cyclopentadiene, cis and/or trans 1,3 pentadienes (Pips), Isoprene, 2-methylbutene-1, 2-methyl-butene-2, pentene, hexane and similar hydrocarbon molecules containing up to 6 carbons. These compounds are used in wide variety of industries to make chemicals, polymers, rubbers etc.

In various embodiments, apparatuses for obtaining streams consisting of single component or components necessary for making specific chemicals and polymers are disclosed. The apparatuses comprise: distillation columns, a compressor, heat exchangers for reboiler and condenser, pumps for pumping product, reflux and water, vessels for keeping the overhead liquid and circulating cooling water.

An embodiment of the invention is directed to a process for the production of purified unsaturated hydrocarbon stream such as ethylene, propylene, butylenes, 2 methyl butene-1, 2 methyl butene-2, Isoprene, cis and trans pentene, Hexene or similar using a system that employs a specific arrangement of distillation column and compressor and operating conditions to reduce the energy consumption, capital investment while at the same time achieving a stable operation. The process employs a low pressure distillation column used as a stripping column (A) and a high pressure distillation column used as a rectification column (B). An overhead compressor (C) compresses the low pressure distillation column overhead, which is introduced to the bottom of the high pressure rectification column as a stripping medium. High pressure liquid from the bottom of the high pressure rectification column is de-pressured to low pressure column conditions through a JT valve or equal and is introduced to the top of low pressure stripping column. Cooling water with or without anti freeze depending on the application, is stored in a vessel and is pumped and circulated through the reboiler or condenser and condenser or reboiler in a sequential manner such that one provides the heat duty required for the other. The overhead vapor from the high pressure rectification column is condensed, stored in a vessel is refluxed and pumped as pure unsaturated product stream. The liquid from the bottom of the low pressure stripping column is pumped as product stream and contains predominantly the saturated hydrocarbon.

In various embodiments of the apparatuses, the distillation column consists of a vessel with trays or packing as internals and may contain partition plates or heads to separate the high pressure and low pressure zone or may implement two columns physically separated from each other and or mounted on top of each other.

In various embodiments the apparatuses, the distillation columns are operated at pressure ratios between 1.5 and 3.0 consistent to keep the system in heat balance and to provide reasonable temperature gradient for heat transfer between the low pressure stripping column reboiler and high pressure rectification column condenser, unlike the traditional distillation columns where both the stripping and rectification occurs at same pressure.

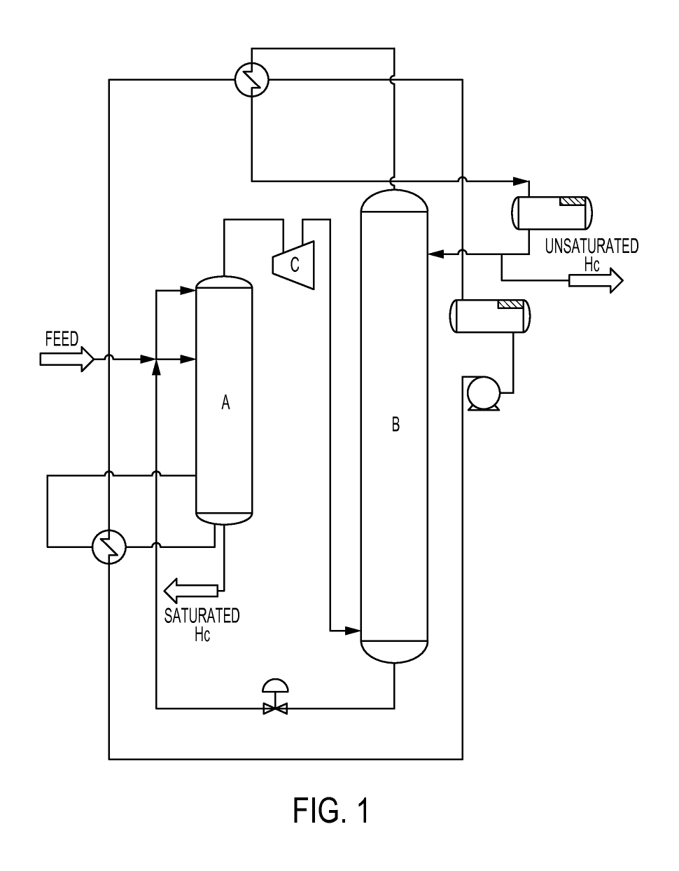

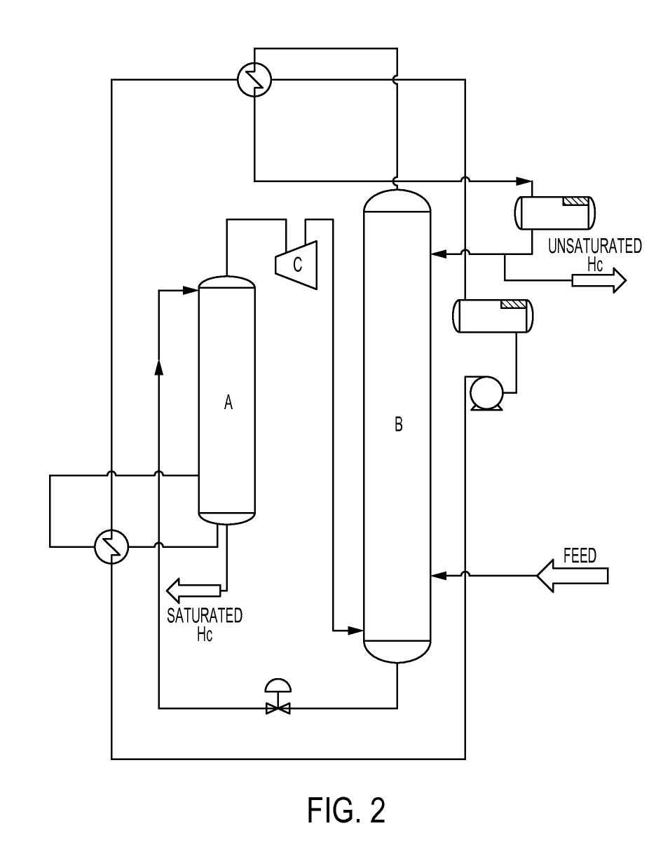

FIG. 1 shows an illustrative unsaturated and saturated hydrocarbon separation system with low pressure stripper feed point. This figure illustrates the coupling of low pressure stripping column (A) and high pressure rectification column (B) using a direct coupled compressor (C). The compressor is operated to have a pressure ratio of 1.5 to 3 depending on the carbon number in the feed. The feed to the unit can be introduced either in the low pressure section (FIG. 1) or in the high pressure section (FIG. 2) depending on the carbon numbers and the energy consumption in the compressor.

In various embodiments, the arrangement shown in FIG. 1 and FIG. 2 is operated such that the liquid recycle from the high pressure rectification to low pressure stripping acts as an operating variable for varying feed compositions. The liquid recycle can be adjusted to compensate the energy difference between the low pressure column reboiler and high pressure column condenser such that the heat exchanger heat transfer areas are optimum for circulating cooling water system.

In various embodiments, the arrangement shown in FIG. 1 and FIG. 2 can be used in combination with a selective hydrogenation unit to saturate the alkynes containing 2-6 carbons.

As shown in the Table below, the heat integrated distillation system provides a process that consumes less energy than traditional prior art systems.

TABLE 1

| | | Scheme 1 | Scheme 2 | Scheme 3 |
|---|---|---|---|---|
| Description -Title | Units | Traditional | Heat Pump | Heat integrated |
| | | Splitter | System | Distillation System |
| No of Columns | | 1 | 1 | 2 |
| No of Pumps | | 3 | 2 | 3 |
| No of Compressors | | 0 | 1 | 1 |
| No of Exchangers | | 2 | 3 | 4 |
| No of Vessels | | 1 | 1 | 1 |
| Main Column | | | | |
| Top Pressure | Kg/Cm2g | 14.8 | 3.54 | 12.2 |
| Bottom Pressure | Kg/Cm2g | 16.5 | 5.3 | 13.4 |
| No. Of Stages | | 147 | 143 | 126 |
| Top Temp | ° C. | 37 | −3.2 | 30 |
| Bottom Temp | ° C. | 51 | 9.5 | 38 |
| Secondary Column | | | | |
| Top Pressure | Kg/Cm2g | NA | NA | 5 |
| Bottom Pressure | Kg/Cm2g | NA | NA | 5.25 |
| No. Of Stages | | NA | NA | |
| Top Temp | ° C. | NA | NA | 7.3 |
| Bottom Temp | ° C. | NA | NA | 12.1 |
| Main Column reboiler Duty | MMKCAL/h | 10.37 | 7.7 | 0 |
| Utilty Consumption (LP Steam) | Kg/H | 20740 | 0 | 0 |
| Main Column Condenser Duty | MMKCAL/h | −10.38 | −8.3 | −13.26 |
| Utilty Consumption (CW) | M3/h | 1297.5 | 0 | 0.0 |
| Secondary Column reboiler Duty | | NA | NA | 11.57 |
| Utilty Consumption | | NA | NA | 0 |
| Secondary Column Condenser Duty | | NA | NA | 0 |
| Utilty Consumption | | NA | NA | 0 |
| Compressor - Compression Ratio | | NA | 4.54 | 3 |
| Polytropic work (Electricity) | KW | NA | 2140 | 1858 |
| Other Exchangers | MMKCAL/h | NA | 2.324 | 0 |
| Utility Cooling Water | M3/h | NA | 290.5 | 0.0 |
| Feed Composition | C3═/C3 | 70/27 | 70/27 | 70/27 |
| Propylene Purity | % | 99.5 | 99.5 | 99.5 |
| Propylene Recovery | % | 98.3 | 98.3 | 98.4 |
| Parameter | | Traditional | Heat Pump | Heat Integrated |
| LP Steam, kg/h | | 20740 | 0 | 0 |
| CW, m\hat{ }3/h | | 1298 | 213 | 0 |
| Electricity, KW | | N/A | 1607 | 1570 |
| Reboiler Heat, Req. MMKcal/hr | | 10.4 | 8.8 | 7.9 (*) |
| Use of Existing Shell | | N/A | Y | Y |

(*) No external utility required.

Some of the advantages of the foregoing inventive system include:
1. Self Serving separation system
2. Zero continuous external steam Consumption
3. Zero continuous external cooling water Consumption
4. Exploits the natural pressure behavior of components in stripping and rectification section
5. Stripping and rectification sections of the distillation column are operated at their best efficiency point compared to traditional distillation
6. Lower electricity consumption compared to a mechanical vapor recompression (MVR) system
7. Stable and ease to operate compared to MVR system
8. Less complexity compared to heat integrated distillation column.

From the foregoing description, one skilled in the art can easily ascertain the essential characteristics of this disclosure, and without departing from the spirit and scope thereof, can make various changes and modifications to adapt the disclosure to various usages and conditions. The embodiments described herein above are meant to be illustrative only and should not be taken as limiting of the scope of the disclosure, which is defined in the following claims.

What is claimed is:

1. A method for producing a unsaturated hydrocarbon stream comprising 2 to 6 carbons, the method comprising employing a low pressure distillation column used as a stripping column and a high pressure distillation column used as a rectification column.

2. The method of claim 1, wherein the feed point is at the low pressure side.

3. The method of claim 1, wherein the feed point is at the high pressure side.

4. The method of claim 1, wherein the low pressure column and high pressure column are connected by an overhead compressor.

5. The method of claim 1, wherein the distillation columns are operated at pressure ratios between 1.5 and 3.0.

6. The method of claim 1, wherein the low pressure column operates at a pressure range of 12.2 to 13.4 Kg/Cm2g.

7. The method of claim 1, wherein the high pressure column operates at a pressure range of 5 to 5.25 Kg/Cm2g.

8. The method of claim 1, wherein the low pressure column operates at a temperature range of 30 to 38° C.

9. The method of claim 1, wherein the high pressure column operates at a temperature range of 7.3 to 12.1° C.
